(19) United States
(12) Patent Application Publication
(10) Pub. No.: US 2024/0385335 A1
(43) Pub. Date: Nov. 21, 2024

(54) ASSEMBLY FOR DETECTING SCATTERED RADIATION AND DETECTION AND VISUALIZATION SYSTEM COMPRISING SAID DETECTION ASSEMBLY

(71) Applicant: TRIXELL, MOIRANS (FR)

(72) Inventor: Jean-Michel VIGNOLLE, MOIRANS (FR)

(21) Appl. No.: 18/662,907

(22) Filed: May 13, 2024

(30) Foreign Application Priority Data
May 17, 2023 (FR) 2304953
May 17, 2023 (FR) 2304954

(51) Int. Cl. G01T 1/08

(52) U.S. Cl. CPC G01T 1/08

(57) ABSTRACT

An assembly for detecting scattered rays includes an X-ray filtering device configured to spatially filter the X-rays as a function of their direction of emission; and an imager assembled facing the filtering device so as to receive the X-rays filtered by the filtering device, the imager being configured to produce a radiographic image from the X-rays filtered by the filtering device; the filtering device comprising several plates, the plates comprising a material capable of absorbing the X-rays, the plates being oriented so as to move away from a centre of the filtering device in the direction going from the imager to the filtering device.

### CROSS-REFERENCE TO RELATED APPLICATIONS

This application claims priority to foreign French patent application Nos. FR 2304954 and FR 2304953, filed on May 17, 2023, the disclosures of which are incorporated by reference in their entireties.

### FIELD OF THE INVENTION

The invention relates to an assembly for detecting scattered radiation and to a detection and visualization system comprising said detection assembly.

### BACKGROUND

The invention is applicable in various fields, for example in X-ray imaging used notably in the medical sector.

The X-rays emitted in certain directions may be at least partly scattered in other directions that are not targeted. Thus, the X-rays emitted initially toward one or more zones are also on other zones not initially targeted. This is the case for example in interventional radiology in which an X-ray generator sends X-rays to a zone of the body of the patient. A part of the X-rays directed toward the patient is absorbed by the latter and another part of these X-rays is scattered by the patient and by the objects located in proximity and which are touched by these rays. The scattered rays go off again in all directions. The medical personnel are then exposed to this scattered radiation, notably when participating in the surgical intervention. The scattered radiation absorbed by the medical personnel during an intervention is not dangerous because the dose received is low, but repeat exposure during multiple interventions may be dangerous.

The medical personnel (and more generally any user exposed to the X-rays) must therefore be protected from the X-rays. To do this, three main protection measures are implemented in the medical sector:
wearing an individual dosimeter, for example on the chest, the dosimeter giving an approximate indication of the total dose received by the person over time;
wearing protective clothes, for example made of lead, attenuating the rays received;
the medical personnel is trained about this risk and the ways of minimising it.

These measures are not ideal. The dosimeter, being a small object, makes it possible to measure the radiation received only in the zone of the body onto which it is fixed and does not therefore register radiation received by other parts of the body of the personnel. The protective clothes, for their part, are heavy and uncomfortable. The medical personnel are therefore reluctant to wear them. Finally, the medical personnel are not always trained, as explained in the article [Badera 2022], and even when they are trained, given that the X-rays are invisible, the personnel do not sufficiently register the danger.

In this context, it may be desirable to detect the X-rays, notably the scattered X-rays. One objective can be notably that of providing better protection from the X-rays and/or protecting the surrounding equipment. It can also be desirable to make the scattered X-rays visible in order for the user, observing the danger, to be able to protect themself therefrom more effectively.

### SUMMARY OF THE INVENTION

The invention aims to propose a detection assembly that makes it possible to detect the X-rays scattered in an environment, and a system that makes it possible to detect and visualize the scattered X-rays.

To this end, it proposes an assembly for detecting scattered rays comprising:
an X-ray filtering device configured to spatially filter the X-rays as a function of their direction of emission; and
an imager assembled facing the filtering device so as to receive the X-rays filtered by the filtering device, the imager being configured to produce a radiographic image from the X-rays filtered by the filtering device.

The filtering device comprises several plates, said plates comprising
a material capable of absorbing the X-rays.

The plates comprise a first series of plates and a second series of plates, the first series of plates comprising first plates following one another in a first direction, the second series of plates comprising second plates following one another in a second direction. The first direction and the second direction are orthogonal to one another.

The plates are oriented so as to move away from a centre of the filtering device in the direction going from the imager to the filtering device. Thus, the first plates are oriented so as to move away from the centre of the filtering device in the direction going from the imager to the filtering device, and the second plates are oriented so as to move away from the centre of the filtering device in the direction going from the imager to the filtering device.

The filtering device makes it possible, by its configuration in the form of plates comprising an absorbent material and having particular orientations, to form a radiological image on the imager which sees only the X-rays arriving in certain directions. The filtering device eliminates (or absorbs) the X-rays arriving in a direction normal to a plane of the imager and lets through the X-rays in other directions which arrive thus on the imager.

Furthermore, the invention makes it possible to detect radiation over a relatively wide field. Indeed, the orientation of the plates moving away from the centre of the filtering device, i.e. toward the outside of the filtering device, makes it possible to detect the X-rays over a wide angle.

Particularly convenient preferred features of the detection assembly according to the invention are presented hereinbelow.

At least two of the first plates are not mutually parallel and at least two of the second plates are not mutually parallel.

Spaces separate the plates from one another.

The filtering device comprises portions, called transparent portions, disposed between the plates. The transparent portions are produced in a material that is transparent to the X-rays.

The imager is curved.

The imager has a hemispherical form.

The imager is flat.

The filtering device and the imager have the same general form.

According to another aspect, the invention relates also to a detection and visualization system comprising:
a detection assembly having at least one of the preceding features;
an optical camera having the same field of view as the imager, the optical camera being configured to produce an optical image; and
a reading and display device connected to the imager and to the camera, the reading and display device being configured to read the image received from the imager and the optical image received from the camera, superimpose the radiographic image and the optical image, and display the superimposition of the radiographic image with the optical image.

The superimposition of the X image with the optical image makes it possible to render the X-rays visible and identify their location and possibly other information (intensity for example). An observer can see where the zones emitting X-rays are located on the optical image.

The solution provided by the invention thus makes it possible to visualize the sources of scattered X-rays likely to irradiate a user and medical personnel, or also X-rays arriving on a user and re-emitted thereby, for example. By virtue of the system of the invention, the visualization can be done in real time. This allows the user to register the moment of danger. The user thus made aware would then take the necessary protection measures.

According to another aspect, the invention can relate to a method for detecting and visualizing X-rays comprising the following steps:
filtering X-rays by means of an X-ray filtering device;
producing an image, called X image, from the X-rays filtered by the filtering device, the X image being produced by means of an imager of X-rays assembled facing the filtering device so as to receive the X-rays filtered by the filtering device;
producing an optical image from an optical camera having the same field of view as the imager;
reading the X image and the optical image by means of a reading and display device connected to the imager and to the camera;
superimposing the X image and the optical image by means of the reading and display device; and
displaying the superimposition of the X image with the optical image by means of the reading and display device.

The filtering device can have at least one of the features previously described.

### BRIEF DESCRIPTION OF THE DRAWINGS

Other features and advantages of the invention will become apparent from the following description with reference to the attached drawings, given as nonlimiting examples:

FIG. 1 schematically represents a radiology room comprising several detection and visualization systems according to an embodiment of the invention;

FIG. 2 individually represents the detection and visualization system of FIG. 1, said system comprising a detection assembly;

FIG. 3 represents the detection assembly with an optical camera;

FIG. 4 represents a filtering device of the detection assembly according to a first embodiment;

FIG. 5 represents the filtering device according to a second embodiment; and

FIG. 6 represents the detection assembly according to another embodiment of the invention with the optical camera.

### DETAILED DESCRIPTION

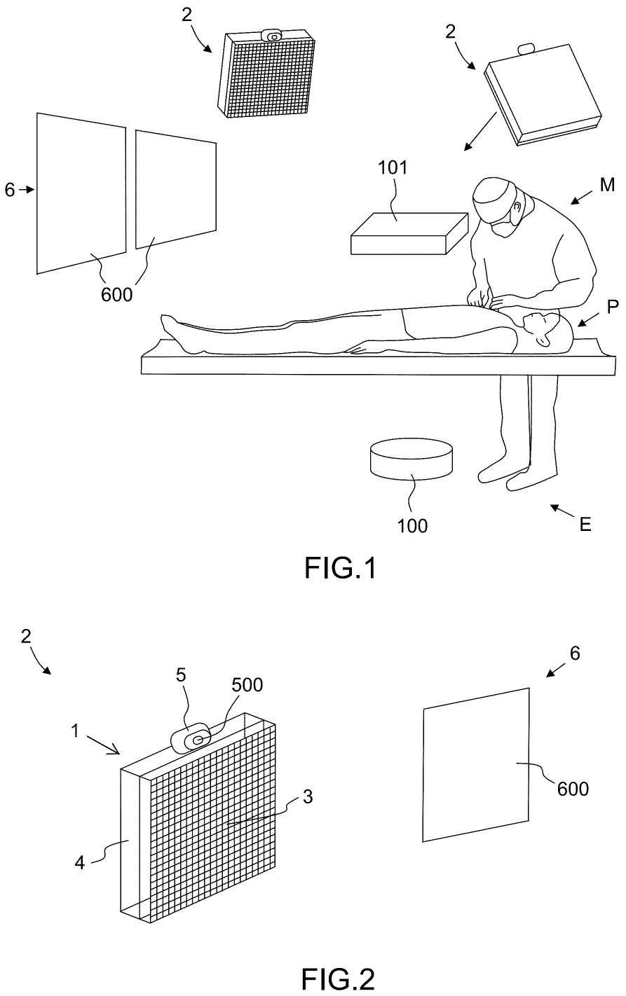

FIG. 1 schematically represents a radiology room in which there is a patient P and a member of medical personnel M. FIG. 1 also illustrates a radiological assembly E directed toward the patient P. The radiological assembly comprises a generator 100 of X-rays and a detector of X-rays 101. The patient P is placed between the generator 100 of X-rays and the detector 101.

The generator 100 of X-rays is configured to generate a beam of X-rays. The generator 100 of X-rays and the detector 101 are positioned with respect to one another in such a way that the X-rays emitted by the generator 100 of X-rays are picked up by the detector 101.

The detector 101 is a traditional image detector. The image detector notably comprises a sensor. The sensor is for example produced on a first substrate. The first substrate comprises a set of pixels organised in a matrix on rows and columns. The matrix can comprise any number of rows and columns thus forming pixels. The matrix forms a geographic zone on the first substrate. The set of the pixels is configured so as to generate signals as a function of radiation arriving on the detector 101.

The pixels comprise a photosensitive zone delivering a current of electrical charges as a function of the stream of photons that it receives, and an electronic circuit for processing this current. The photosensitive zone generally comprises a photosensitive element, or photodetector, which can for example be a photodiode, a photoresistor or a phototransistor. The electronic circuit is for example composed of switches, capacitors, resistors, and downstream of which there is an actuator. The assembly composed of the photosensitive element and the electronic circuit makes it possible to generate electrical charges and to collect them.

FIG. 1 also represents two X-ray detection and visualization systems 2 according to an embodiment of the invention. The X-ray detection and visualization system 2 according to this exemplary embodiment is also represented individually in FIG. 2.

The detection and visualization system 2 comprises a scattered X-ray detection assembly 1, an optical camera 5 and a reading and display device 6.

The detection assembly 1 comprises an X-ray filtering device 3 and an X-ray imager 4.

The X-rays are a form of high-frequency electromagnetic radiation, the wavelength of which lies approximately between 5 picometres and 10 nanometres. The energy of these photons goes from a few eV (electron-volts), to several tens of MeV. One of the main properties of the X-rays is that they easily penetrate the “soft matter” (solid matter that is not very dense and composed of light elements such as carbon, oxygen and nitrogen) and are easily absorbed by the “hard matter” (dense solid matter composed of heavy elements). This property makes the medical imaging possible, with the rays passing through the flesh and being stopped by the bones.

The filtering device 3 is configured to filter X-rays.

In an interventional radiology situation, the filtered X-rays are typically derived from the rays scattered when a patient receives rays by the generator 100 of X-rays. In particular, the filtered X-rays are derived from the “primary” scattered rays, i.e. those scattered by the patient receiving rays emitted by the generator 100 of X-rays and by the objects located in proximity and which are touched by the rays sent by the generator 100 of X-rays. The filtered X-rays can also be “secondary” scattered X-rays, i.e. rays derived from the scattering by the objects or the personnel in proximity, X-rays emitted by “primary” scattering.

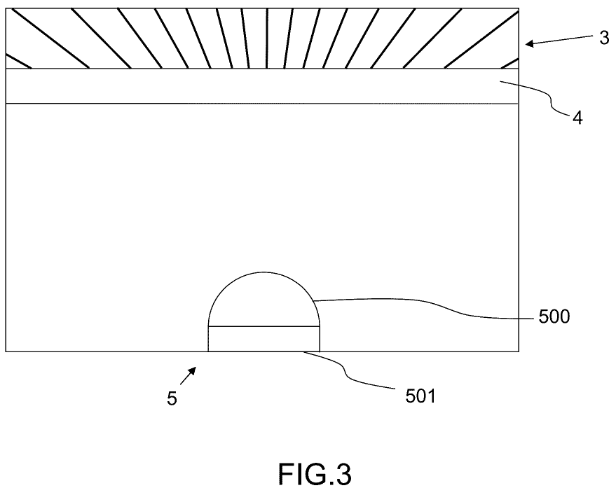

The filtering device 3 is assembled facing the imager 4, as represented schematically in FIGS. 1 to 3.

Preferably, the distance separating the filtering device 3 and the imager 4 is small. Preferably, the distance separating the filtering device 3 and the imager 4 is less than 30 cm. This allows good detection of the X-rays even when the stream thereof is weak.

The filtering device 3 is, here, fixed to the imager 4.

The filtering device 3 and the imager 4 have the same general form. The part or surface of the filtering device 3 assembled with the imager 4 has the same dimensions as the imager 4. The imager 4 has for example a generally rectangular form. The filtering device 3 also has rectangular outlines.

The imager 4 is flat in the embodiment of FIGS. 1 to 3. The filtering device 3 is also flat. The imager 4 and the filtering device 3 have, in FIG. 1, a square form. For example, the imager 4 and the filtering device 3 each have a square form with 20 cm sides.

The filtering device 3 and the imager 4 are parallel to one another.

The imager 4 is configured to produce a radiographic image or X image from the X-rays filtered by the filtering device 3.

The imager 4 is for example similar to the X-ray detector 101. The imager 4 notably comprises a sensor. The sensor is for example produced on a first substrate. The first substrate comprises a set of pixels organised in a matrix on rows and columns. The matrix can comprise any number of rows and columns thus forming pixels. The matrix forms a geographic zone on the first substrate. The set of pixels is configured so as to generate signals as a function of radiation arriving on the imager 4.

The pixels are sensitive to the X-rays and deliver an electrical signal (notably an electrical charge), the level of which is a function of the intensity of the X-rays. In other words, the pixels comprise a photosensitive element, or photodetector, which can for example be a photodiode, a photoresistor or a phototransistor. The electrical signals from the different pixels are collected by reading circuits of the imager 4 during a phase of reading of the matrix, then digitized so that they can be processed and stored to form the radiographic image.

The photosensitive elements make it possible to detect visible or near-visible electromagnetic radiation. These elements are not sensitive, or not very sensitive, to the radiation incident to the detector. A radiation converter, called scintillator, is then frequently used, which converts the incident radiation, for example X radiation, into radiation in a band of wavelengths to which the photosensitive elements present in the pixels are sensitive. One alternative consists in producing the photosensitive element in another material carrying out the direct conversion of the X radiation into electrical charges. This is the case for example of the matrices in which a first pixelated substrate produced in cadmium telluride (CdTe) is connected pixel-by-pixel to a CMOS reading circuit which therefore no longer has the detection function.

The imager 4 is preferably very sensitive and not noisy. This is expressed conventionally by the value of the NED (“Noise Equivalent Dose”, the dose for which the noise of X is equal to the electronic noise of the imager 4). For example, the signal received by the imager 4 in the case of an aperture of 1 cm² to 20 cm², and of a dose of X-rays sent to the patient by a source of X-rays of intensity 10 mAs situated at 1 m from the patient, with an energy of 80 kV to 120 kV, could be only 0.01 to 0.05 nGy (nanoGray), which is very little. The imager 4 therefore has the capacity to produce legible images with doses of this order of magnitude, and its NED is typically less than or equal to 0.01 nGy. If the NED of the imager 4 is higher, the images from the imager 4 are averaged over time (temporal averaging, for example of 100 successive images) and/or in space (spatial averaging, for example by blocks of 10×10 pixels), which limits the temporal and/or spatial resolution, but brings the NED to the desired level.

The filtering device 3 comprises a material capable of absorbing the X-rays. The material capable of absorbing the X-rays is for example lead, copper or tungsten. Copper has the advantage of being inexpensive, rigid, and does not present any danger to the environment.

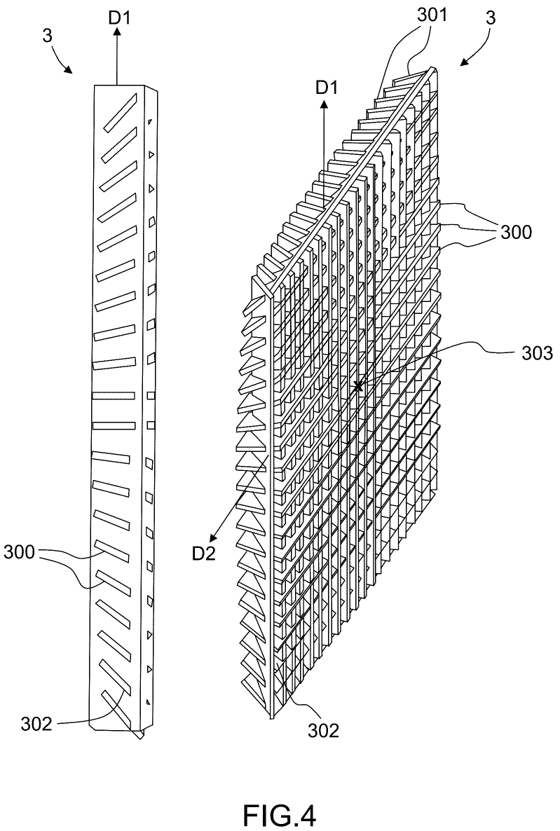

The filtering device 3, schematically represented in FIGS. 1 and 2 according to an embodiment, is represented individually and from different viewing angles in FIG. 4.

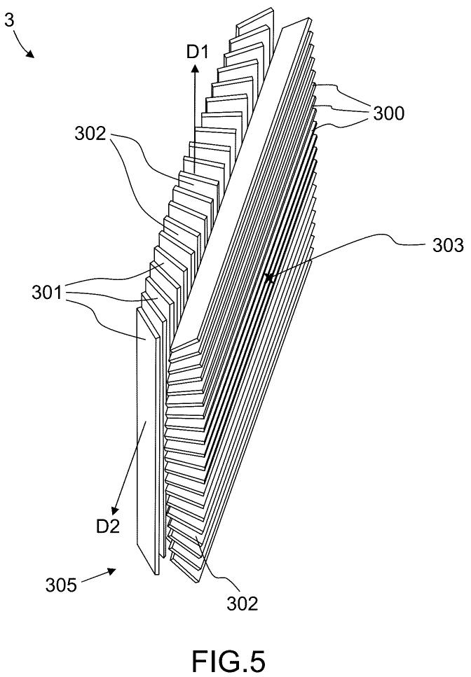

The filtering device 3 comprises several plates 300, 301 (represented in FIGS. 4 and 5). The other figures represent the filtering device 3 very schematically.

The plates 300, 301 comprise or are produced in a material capable of absorbing the X-rays (for example lead, copper or tungsten). The plates 300, 301 are oriented so as to absorb the X-rays in several directions.

Each plate has a rectangular form. Each plate thus has a longitudinal dimension or length, a lateral dimension or width, and a transverse dimension or thickness.

The plates 300, 301 are, here, separated by spaces 302. In other words, a gap separates adjacent plates 300, 301 from one another.

According to a variant embodiment, the plates 300, 301 are not separated by an empty space. The filtering device 3 comprises portions of material disposed between the plates 300, 301. The portions of material are produced with a material that is transparent to the X-rays. Preferably, the portions of material (also here called transparent portions) are rigid. This makes it possible to keep the plates 300, 301 firmly in place. The portions of material are for example portions of polyurethane foam.

The plates 300, 301 comprise a first series of plates and a second series of plates. The first series of plates comprises first plates 300 that follow one another in a first direction D1. The second series of plates comprises second plates 301 that follow one another in a second direction D2. The first direction D1 and the second direction D2 are distinct, here orthogonal to one another.

A space 302 here separates each of the first plates 300. Likewise, a space 302 here separates each of the second plates 301.

The plates 300, 301 are oriented so as to move away from a centre 303 of the filtering device 3. In particular, the first plates 300 are oriented so as to move away from the centre 303 of the filtering device 3 and the second plates 301 are oriented so as to move away from the centre 303 of the filtering device 3. The plates 300, 301 move away from the centre 303 in the direction going from the imager 4 to the filtering device 3. In other words, the plates 300, 301 are oriented toward the outside of the filtering device 3. The plates 300, 301 thus configured, when the filtering device 3 is used with a radiological assembly, diverge from a generator of X-rays.

The plates 300, 301 are oriented so as to filter the X-rays in several directions. The plates 300, 301 are oriented according to an angle of orientation that is non-zero with respect to the plane created by the first direction D1 and the second direction D2.

The plates 300, 301 are each oriented according to a predefined angle of orientation such that, for given plates 300, 301, the X-rays arriving in certain directions are blocked by said given plates 300, 301. Said given plates 300, 301 absorb the X-rays arriving in said directions. The X-rays arriving in a different direction pass through.

When the filtering device 3 comprises spaces 302 between the plates 300, 301, the X-rays pass through when they arrive in the space between two plates 300, 301. In other words, the X-rays which pass through are those which do not arrive on a plate. In other words, the X-rays arriving in a given direction are absorbed by the plates 300, 301 except in a given part of the filtering device 3 in which said X-rays pass between two plates 300, 301 and land on a given zone of the imager 4.

The operation of the filtering device 3 is similar when it comprises a material transparent to the X-rays in place of the empty spaces 302 between the plates 300, 301.

By virtue of this configuration of the filtering device 3, each zone of the imager 4 sees only one direction of X-rays. The direction of X-rays seen by each zone of the imager 4 depends at least on the orientation of the plates 300, 301 of the filtering device 3.

The angle of orientation is modified along the filtering device 3. Several plates 300, 301 can have the same angle of orientation. The angles of orientation of the plates 300, 301 vary so as to absorb the X-rays in certain directions and to allow them to pass in other directions.

Preferably, at least two of the first plates 300 are not parallel to one another and at least two of the second plates 301 are not parallel to one another. In other words, at least two of the first plates 300 have angles of orientation that are different from one another and at least two of the second plates 301 have angles of orientation that are different from one another.

The filtering device 3 as described previously forms a lens for X-rays. The filtering device 3, through the use of several radio-opaque plates 300, 301, “cuts” the X-rays. The final radiographic image obtained by the imager 4 is an assembly of the images obtained by the reception of the X-rays “cut” by the filtering device 3. The assembly of the images is performed in mosaic fashion. The assembly of the images is similar to that done by insects that have compound eyes.

The number of plates 300, 301, their thickness and their orientation define the filtered image. The number of plates 300, 301, their thickness and their orientation are predefined and dimensioned as a function of requirements. These parameters define the separation (or the space when there is one) between the plates 300, 301.

The plates 300, 301 of the filtering device 3 have a thickness of between 0.1 mm and 5 mm, preferably of approximately 1 mm.

The width of each plate 300, 301 is between 1 mm and 10 cm, preferably equal to 1 cm.

The length of each plate 300, 301 depends on the size of the imager 4 used. The length of each plate is less than or equal to the length of the imager 4, and preferably equal to the length of the imager 4. The length of each plate is between 1 cm and 50 cm, preferably equal to 20 cm.

The angle of orientation of the plates 300, 301 is between −90° and 90°, preferably between −60° and 60°.

The number of plates 300, 301 is between 10 and 1000, preferably approximately 100.

The filtering device 3, flat in the exemplary embodiments of FIGS. 1 to 5, has a depth, i.e. a total thickness, of between 1 and 10 cm. The filtering device 3 is thus very compact.

The X image is also a function of the number of pixels of the imager 4 located facing the spaces 302 between the plates 300, 301. Several pixels are preferably facing each space 302 between the plates 300, 301.

The filtering device 3 can be produced in a single piece, as is the case in FIG. 4.

In a variant represented in FIG. 5, the filtering device 3 is produced in two parts. The filtering device 3 comprises a first part 304 and a second part 305. The first part 304 comprises the first series of first plates 300. The second part 305 comprises the second series of second plates 301. The plates are each oriented according to a predefined angle of orientation, in a way similar to the embodiment of FIG. 4.

The first part 304 and the second part 305 can be superimposed as in FIG. 5.

In a variant that is not represented, the first part 304 and the second part 305 can be intersected.

The production of the filtering device 3 in two parts is more economical than production in a single piece.

The filtering device 3 can be manufactured by 3D printing, by moulding, or by any other means which makes it possible to obtain the desired geometry and forms.

The filtering device 3 is preferably disposed at a distance from the zone to be imaged of between 10 cm and 5 m, preferably of the order of 2 m. The smaller the distance, the better the quality of the radiographic image obtained.

However, it should be noted that the imager 4 has the following advantage. The signal received by a pixel does not decrease with the distance between the imager 4 and the zone to be imaged, provided that the zone to be imaged is fairly large and always covers the pixel. In fact, the X-ray signal decreases in 1/R², R being the distance between the imager and the zone to be imaged, but the surface of the zone which is imaged in a pixel increases as R². The two effects compensate one another, and the signal is constant.

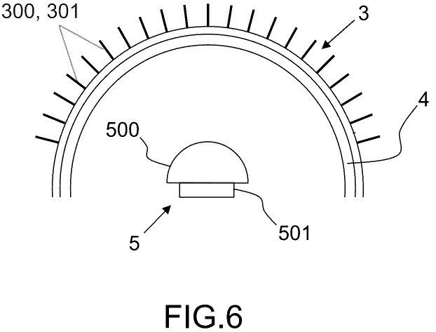

The imager 4 can have a different form, for example the imager 4 can be curved. The imager 4 can have a dome or hemispherical form, as illustrated in FIG. 6. The filtering device 3 has a similar form, here a dome form.

The imager 4 and the filtering device 3 have the same features as previously described for the imager 4 and the filtering device 3 that are flat.

In particular, the filtering device 3 comprises several plates 300, 301 moving away from the centre 303 of the filtering device 3 in the direction going from the imager 4 to the filtering device 3.

The filtering device 3 is also here fixed to the imager 4.

The dome or hemispherical form allows the imager 4 to pick up more radiation compared to the flat form.

The dome can typically have a diameter of between 10 cm and 40 cm, for example 20 cm. The height can be between 5 cm and 20 cm, for example 10 cm.

The radiographic image obtained depends, as explained above, on the configuration of the filtering device 3. The radiographic image is also a function of the number of pixels of the imager 4 located facing the spaces 302 between the plates 300, 301 or the portions made of material that is transparent to the radiation. Several pixels are preferably facing each space between the plates 300, 301, or each portion made of material that is transparent to the X radiation.

Each pixel has dimensions of around a millimetre, or of a few millimetres. These dimensions are sufficient for the intended applications which do not require a high image resolution.

The optical camera 5, visible in FIGS. 1, 2, 3 and 5, is configured to produce an optical or visible image. The camera 5 comprises a lens 500 and a CCD or CMOS photographic sensor 501.

The imager 4 and the lens 500 of the optical camera 5 are directed toward the same point or the same zone to be captured or imaged. The optical camera 5 has substantially the same field of view as the imager 4. For example, the imager 4 and the lens 500 of the camera 5 can be directed toward the zones or the people exposed to the X-rays.

The optical camera 5 is disposed in a plane parallel to the imager 4. The optical camera 5 can be disposed above the imager 4 (FIG. 1). The optical camera 5 can be disposed at the centre or focal point of the dome (FIG. 6) or other curved form.

The reading and display device 6 is connected to the imager 4 and to the camera 5. The reading and display device 6 is configured to read the radiographic image received from the imager 4 and the optical image received from the camera 5, superimposing the radiographic image and the optical image, and displaying the superimposition of the radiographic image with the optical image.

The reading and display device 6 comprises means for connection to the imager 4 and to the optical camera 5. The connection means are for example connector systems or wireless connection means.

The reading and display device 6 comprises an image reading and processing system. The reading and display device 6 can comprise a computer for the image processing. The image processing can be performed by software that makes it possible to combine the optical image and the radiographic image.

The reading and display device 6 further comprises a display 600, such as a monitor or screen for displaying the superimposition of the radiographic image and of the optical image. The display 600 is connected to the image reading and processing system.

The superimposition of the radiographic image with the optical image shows the X-rays by displaying them on the optical image. In other words, the scattered X-rays can be visualized with the point where they are scattered by virtue of the superimposition with the optical image.

The X-rays can be displayed in a colour that is different from the optical image. The optical image can for example be in black and white and the X-rays in a different colour.

Information of interest can also be displayed, such as the intensity of the X-rays. The numeric values of the intensity can be displayed and/or the X-rays can be displayed in different colours depending on their intensity.

The zones scattering the X-rays, and possibly other information such as their intensity, are displayed overprinted on the optical image. The user can see this information appear in real-time on the display 600. This could for example urge them (notably in the case of medical personnel) to protect themselves from the X-rays.

One or more detection and visualization systems 2 can be used to detect and visualize the zones scattering X-rays. This makes it possible to enhance the detection and the visualization of the zones scattering X-rays. FIG. 1 represents two detection and visualization systems 2.

In an exemplary embodiment, one common display 600 can be used for several detection and visualization systems 2.

The method for detecting and visualizing X-rays proceeds as follows. The X-rays are filtered by means of an X-ray filtering device 3.

The filtered X-rays are received by the imager 4. The X image is produced by the imager 4 from the X-rays filtered by the filtering device 3.

In parallel or at a different time, the optical camera 5 produces an optical image.

The X image and the optical image are both read by means of the reading and display device 6 connected to the imager 4 and to the camera 5.

The reading and display device 6, for example by means of image-processing software, superimposes the X image and the optical image.

The reading and display device 6 finally displays the superimposition of the X image and of the optical image on the display 4. The reading and display device 6 can display other information, such as the intensity of the X-rays.

The detection assembly according to the invention makes it possible, through a simple structure and configuration, to spatially filter the X-rays as a function of their direction of emission. Through its diverging configuration from the centre of the filtering device, the latter allows the detection assembly to have a wide field or wide angle to pick up the X-rays on the imager.

The detection and visualization system makes it possible to detect and visualize the zones scattering X-rays so as to be be able to protect oneself therefrom for example. The display can be done in real time in order to directly supply the information on the scattering of the X-rays.

### BIBLIOGRAPHY

[Badera 2022]: Al Mohammad, Badera, Monther Gharaibeh, and Maram Al Alakhras. “Knowledge and practice of radiation protection in the operating theater among orthopedic surgeons.” Journal of Medical Imaging 9.6 (2022): 066002.

1. An assembly for detecting scattered rays comprising:
an X-ray filtering device configured to spatially filter the X-rays as a function of their direction of emission; and
an imager assembled facing the filtering device so as to receive the X-rays filtered by the filtering device, the imager being configured to produce a radiographic image from the X-rays filtered by the filtering device,
the filtering device comprising several plates, said plates comprising a material capable of absorbing the X-rays, the plates comprising a first series of plates and a second series of plates, the first series of plates comprising first plates that follow one another in a first direction (D1), the second series of plates comprising second plates that follow one another in a second direction (D2), the first direction (D1) and the second direction (D2) being orthogonal to one another, the first plates being oriented so as to move away from a centre of the filtering device in the direction going from the imager to the filtering device, the second plates being oriented so as to move away from the centre of the filtering device in the direction going from the imager to the filtering device.

2. The assembly according to claim 1, wherein at least two of the first plates are not parallel to one another and at least two of the second plates are not parallel to one another.

3. The assembly according to claim 1, wherein spaces separate the plates from one another.

4. The assembly according to claim 1, wherein the filtering device comprises portions, called transparent portions, disposed between the plates, the transparent portions being produced in a material that is transparent to the X-rays.

5. The assembly according to claim 1, wherein the imager is curved.

6. The assembly according to claim 1, wherein the imager has a hemispherical form.

7. The assembly according to claim 1, wherein the imager is flat.

8. The assembly according to claim 1, wherein the filtering device and the imager have the same general form.

9. Detection and visualization system comprising:
the assembly according to claim 1;
an optical camera having the same field of view as the imager, the optical camera being configured to produce an optical image; and
a reading and display device connected to the imager and to the camera, the reading and display device being configured to read the image received from the imager and the optical image received from the camera, superimpose the radiographic image and the optical image, and display the superimposition of the radiographic image with the optical image.
